(19) United States
(12) Patent Application Publication
(10) Pub. No.: US 2002/0054871 A1
(43) Pub. Date: May 9, 2002

(54) Methods and compositions for use in the treatment of hyperlipidemia

(76) Inventors: Yadong Huang, San Francisco, CA (US); Robert W. Mahley, San Francisco, CA (US); John M. Taylor, San Francisco, CA (US)

Correspondence Address: Bret Field, Bozicevic Field & Francis LLP, 200 Middlefield Road, Suite 200, Menlo Park, CA, 94025, US

(21) Appl. No.: 09/544,910

(22) Filed: Apr. 7, 2000

Related U.S. Application Data
(60) Provisional application No. 60/128,853, filed on Apr. 12, 1999.

(51) Int. Cl. A01K 67/00; A01K 67/033; A61K 49/00; C07H 21/02; C07H 21/04; A61K 39/395; C07K 1/00; C07K 14/00; C07K 17/00; C07K 16/00

(52) U.S. Cl. 424/130.1; 424/9.2; 424/145.1; 424/158.1; 530/350; 530/387.1; 536/23.1; 536/24.5

(57) ABSTRACT

Methods of treating a host suffering from hyperlipidemia resulting from elevated levels of at least one of VLDL and triglycerides are provided. In the subject methods, an effective amount of agent that reduces the level of active apoE, e.g. apoE inhibitor or apoE expression inhibitor, is administered to the host. The subject methods find particular use in the treatment of hosts suffering from Type IV or Type IIb hyperlipidemia. Also provided are non-human transgenic animal models for hyperlipidemia, as well as methods for making and using the subject animal models, e.g. in therapeutic agent screening applications.

### ACKNOWLEDGMENT OF GOVERNMENT SUPPORT

This invention was made with Government support under Grant No. HL47660 and HL51588 awarded by the National Institute of Health. The Government has certain rights in this invention.

### INTRODUCTION

1. Field of the Invention

The field of the invention is hyperlipidemia.

2. Background of the Invention

Hyperlipidemias are conditions of abnormal plasma lipid/lipoprotein/cholesterol levels, and include hypercholesterolemia and hypertriglyceridemia. Hypertriglyceridemia (HTG) is a common inherited disorder of lipid metabolism in humans that is characterized by a proatherogenic lipoprotein profile, including increased plasma triglycerides and very low density lipoproteins (VLDL), and often decreased high density lipoproteins (HDL). Whereas its frequency in the general population is ˜1% (1), HTG occurs in ˜5% of patients surviving a myocardial infarction, indicating an increased risk for atherosclerosis. Investigations of the pathogenesis of HTG have suggested both increased VLDL triglyceride production and reduced VLDL catabolism; however, the molecular mechanism of HTG remains unknown.

Specific types of hyperlipidemias associated with vascular disease include Type IIb and Type IV hyperlipidemias. Type IV hyperlipidemia is characterized by elevated plasma levels of very low density lipoprotein (VLDL). Type IIb hyperlipidemia is characterized by elevated levels of VLDL and low density lipoprotein (LDL).

Because of their link with vascular disease, a number of approaches for controlling hyperlipidemias have been developed. Such approaches include changes in lifestyle, e.g. diet, exercise, and the like, as well drug therapy. Drugs finding use in the management of plasma lipid profiles include: bile acid binding resins; niacin; HMG-CoA reductase inhibitors; fibric acid derivatives, e.g. gemfibrozil; and the like.

Despite the development of the above protocols, there continues to be a need for the identification of new treatment therapies for hyperlipidemias. Of particular interest would be the development of new treatment therapies for Type IIb and Type IV hyperlipidemias, which account for the majority of clinical hyperlipidemic patients.

Relevant Literature

Patent documents of interest include: U.S. Pat. No. 5,767,337 and WO 97/05247.

Other references of interest include: Shimano et al., P.N.A.S. USA (March 1992) 89: 1750-1754; Fan et al., J. Clinical Invest. (May 1998) 10:2151-2164; Huang et al., J. Biol. Chem. (Oct. 9, 1998) 273:26388-26393; Huang et al., J. Biol. Chem. (Jul. 10, 1998) 273:17483-17490; Sullivan et al., J. Biol. Chem. (Jul. 18, 1997) 272:17972-17980; Salah et al., J. Lipid Res. (May 1997) 38:904-912; Cohn et al., Arterioscl. Thromb. Vasc. Biol. (January 1996) 16:149-159; and Taylor & Fan, Fronteirs in Bioscience (Jun. 15, 1997) 2:d298-308.

References of interest providing background information on hyperlipidemia include: Foxton et al., Nursing Standard (Jun. 13, 1998) 12:49-56; Krauss, The American Journal of Medicine (Jul. 6, 1998)105:58S-62S; and Harrison's Principles of Internal Medicine (14^{th} Edition, 1998) pp 2138-2148.

### SUMMARY OF THE INVENTION

Methods of treating a host suffering from hyperlipidemia resulting from elevated plasma levels of at least one of VLDL and triglycerides are provided. In the subject methods, an effective amount of agent that reduces the plasma level of active apoE, e.g. apoE inhibitor or apoE expression inhibitor, is administered to the host. The subject methods find particular use in the treatment of hosts suffering from Type IV or Type IIb hyperlipidemia. Also provided are non-human transgenic animal models of hyperlipidemia, as well as methods for making and using the subject animal models, e.g. in therapeutic agent screening applications.

### BRIEF DESCRIPTION OF THE FIGURES

FIG. 1. Superose 6 chromatography of 100 μl of mouse plasma. Each panel is one representative profile of several analyzed in each group of mice. TC, total cholesterol; TG, triglyceride. The units for apoE3, TC, and TG are mg/dl.

FIG. 2. Correlation of plasma or VLDL apoE with plasma triglyceride levels, VLDL triglyceride production, or VLDL lipolysis. (A) Plasma apoE versus plasma triglyceride levels in apoE3 transgenic mice (r=0.92,p<0.001, n=52). (B) Plasma apoE versus VLDL triglyceride production rates in apoE3 transgenic mice (r=0.91,p<0.001, n=12). (C) Plasma apoE versus plasma triglyceride levels in human subjects (r=0.93,p<0.001, n=38). (D) VLDL apoE versus VLDL lipolysis in human subjects (r=−0.77,p<0.001, n=38).

FIG. 3. Effect of apoE3 and apoCII on the lipolysis of VLDL. (A) Various samples of VLDL (30 μg of triglycerides) were incubated with 1 μg of purified bovine milk LPL (Sigma) or 50 μl of hepatic lipase-transfected cell-conditioned medium for 30 min at 37° C. After incubation, the levels of free fatty acids were determined. Results are the mean ±S.D. of determinations in four mice. (B) Normal mouse VLDL (30 μg of triglycerides) were incubated with various amounts of purified human apoE3 for 30 min at 37° C. and then with 1 μg of bovine milk LPL for another 30 min at 37° C. Results are the mean ±S.D. of determinations in three mice. (C) Various VLDL (30 μg of triglycerides) were incubated first with 16 μg of purified human apoC-II for 30 min at 37° C. and then with 1 μg of bovine milk LPL for another 30 min at 37° C. Results are the mean ±S.D. of determinations in four mice.

FIG. 4. Superose 6 chromatography of 100 μl of human plasma. The cholesterol and triglyceride distributions in the plasma of individual subjects were analyzed as described previously. Each panel is one representative profile of several analyzed in each group of human subjects. (A) Normal control plasma. (B) Plasma from a type IV hyperlipidemic subject. (C) The same plasma in panel B after in vitro lipolysis by LPL. Plasma (300 μl) was incubated with 4 μg of bovine milk LPL for 1 h at 37° C. (D) Normal control plasma supplemented with autologous VLDL before in vitro lipolysis. (E) The same sample as panel D after in vitro lipolysis. The sample was incubated with 4 μg of bovine milk LPL for 1 h at 37° C. (F) The sample from panel D was first incubated with purified apoE3 (at a final concentration of 14 mg/dl) for 20 min at 37° C. and then incubated with 4 μg of bovine milk LPL for another 1 h at 37° C. TC, total cholesterol; TG, triglyceride. The units for apoE, TC, and TG are mg/dl.

FIG. 5 provides Table 1, which shows the lipid and apoE levels in plasma and VLDL from different lines of mice.

FIG. 6. provides Table II, which shows the effect of apoE expression levels of VLDL triglyceride production in McA-RH7777 cells stably transfected with various apoE isoforms.

FIG. 7 provides Table III, which shows the lipid and apoE levels in plasma and VLDL from normal or type IV hyperlipidemic human subjects.

FIG. 8. Correlation of plasma lipids with apoE3 levels. Total cholesterol (TC) and triglycerides (TG) were determined in whole plasma of 21 individual male rabbits (4 nontransgenic and 17 transgenic). The lines represent the best fit curve, as determined by regression analysis. Plasma total cholesterol increased proportionally with increasing levels of apoE3, whereas plasma triglycerides remained unchanged (or slightly decreased) at apoE3 levels <20 mg/dL but increased sharply at apoE3 levels >20 mg/dL.

FIG. 9. Superose 6 chromatography of 200 μL of rabbit plasma. The cholesterol and triglyceride distributions in the plasma of individual male rabbits were analyzed as described previously. Each panel is one representative profile selected from plasma profiles of several rabbits in each group. The bars in panel A indicate the plasma lipoprotein classes. The LDL fraction also contains a small quantity of HDL₁. TC, total cholesterol; TG, triglyceride. The units for apoE3, TC, and TG are mg/dL.

FIG. 10. Correlation of plasma lipoproteins with apoE3 levels. The lines represent the best fit curve, as determined by regression analysis. VLDL, IDL, and LDL cholesterol and triglycerides were calculated from the Superose 6 chromatography profiles of plasma lipoproteins from 14 individual male rabbits by summing the individual fractions (FIG. 9). TC, total cholesterol; TG, triglyceride.

FIG. 11. Effects of apoE3 expression levels on VLDL triglyceride production. A, three male rabbits from each group were injected intravenously with Triton WR1339 after an overnight fast. Plasma triglyceride (TG) concentrations were measured before and at different times after injection. The hepatic VLDL triglyceride production rate was calculated from the slope of the curve. Tg, transgenic. *P<0.001 (t-test) versus nontransgenic. **P<0.001 (t-test) versus apoE3 medium expresser. B, correlation of VLDL triglyceride production with plasma apoE3 levels (linear regression analysis).

FIG. 12. Correlation of plasma apoE3 levels with VLDL and IDL lipolysis. Various samples of VLDL (A) or IDL (B) (30 μg of triglycerides) isolated from 16 individual male rabbits were incubated with 1 μg of purified bovine milk LPL for 30 minutes at 37° C. After incubation, the levels of free fatty acids (FFA) were determined. The lines represent the best fit curve from linear regression analysis.

FIG. 13 Plasma clearance and liver uptake of ¹²⁵I-VLDL. ¹²⁵I-labeled VLDL (5 μg of protein in 100 μL of saline) pooled from 4-5 rabbits from each of three groups were injected into the tail vein of normal mice. Plasma clearance (A) and liver uptake (B) of the ¹²⁵I-VLDL were determined as described previously. Each time point represents the average±SD of determinations in 3 mice.

FIG. 14. Summary of the effects of apoE3 expression levels on apoB-containing lipoprotein metabolism in transgenic rabbits. Compared with nontransgenics, apoE3-low expressers (<10 mg/dL) had a significant increase in VLDL clearance, with slightly increased VLDL production and slightly decreased VLDL lipolysis, leading to slightly decreased VLDL. The enhanced VLDL clearance competes with LDL catabolism via the LDL receptor pathway, leading to a slight to moderate increase in LDL cholesterol. In apoE3-medium expressers (10-20 mg/dL), the small further increase in clearance is just about or not quite sufficient to compensate for the further increase in production and the impairment of lipolysis; thus, VLDL steady-state levels are only slightly higher than in nontransgenics. However, since VLDL lipolysis is not dramatically affected, the overproduced VLDL will be effectively converted to LDL, and together with catabolic competition derived from enhanced VLDL clearance, lead to a dramatic increase in LDL cholesterol. In apoE3 high expressers (>20 mg/dL), the clearance rate increase is not nearly large enough to compensate for the dramatically increased production and the more severely impaired lipolysis, leading to increased VLDL cholesterol and triglycerides. Furthermore, dramatically impaired VLDL lipolysis decreases the number of VLDL particles that transit the lipolytic cascade. Also, since LDL catabolism is already maximally inhibited by the competition of enhanced VLDL clearance, the steady-state levels of LDL do not differ from those in medium expressers. Although IDL are not included in the figure, the effect of apoE3 expression levels on IDL metabolism may be similar to that on VLDL. Increasing apoE3 expression levels enhances IDL clearance, inhibits lipolytic conversion of IDL to LDL (FIG. 13), and stimulates IDL production secondary to an increase in VLDL production.

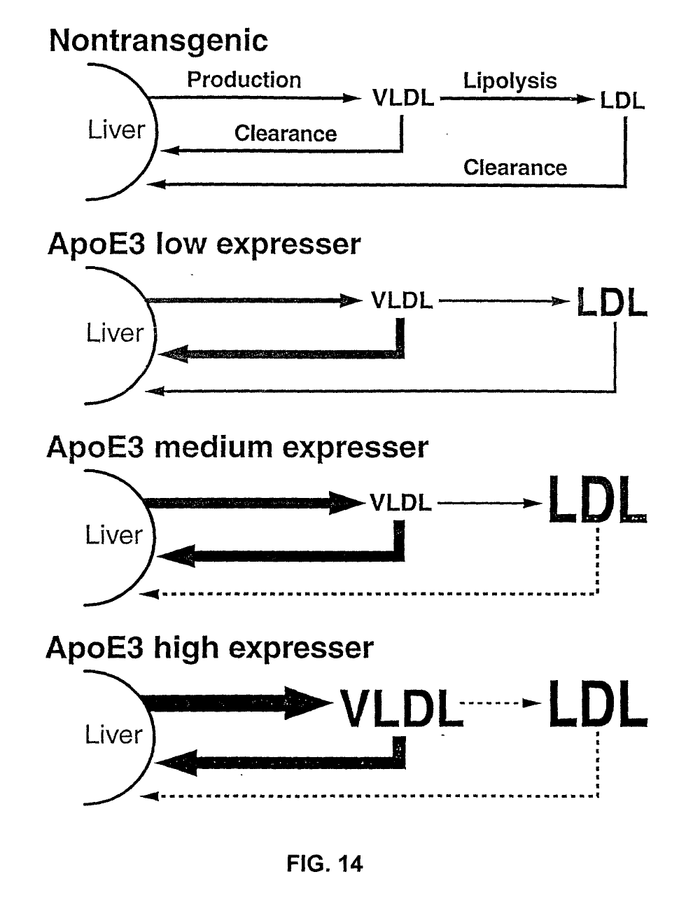

FIG. 15 provides Table IV which shows the plasma lipid levels of ApoE transgenic rabbits.

### DEFINITIONS

The term “transgene” is used herein to describe genetic material which has been or is about to be artificially inserted into the genome of a cell, particularly a mammalian cell for implantation into a living animal.

By “transformation” is meant a permanent or transient genetic change, preferably a permanent genetic change, induced in a cell following incorporation of new DNA (i.e., DNA exogenous to the cell). Where the cell is a mammalian cell, a permanent genetic change is generally achieved by introduction of the DNA into the genome of the cell.

By “transgenic animal” is meant a non-human animal, usually a mammal (e.g., mouse, rat, rabbit, hamster, etc.), having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

A “knock-out” of a gene means an alteration in the sequence of the gene or sequence associated with the gene that results in a decrease of function of the target gene, preferably such that target gene expression is undetectable or insignificant. “Knock-out” transgenics can be transgenic animals having a heterozygous knock-out of a gene or a homozygous knock-out of a gene. “Knock-outs” also include conditional knock-outs, where alteration of the target gene can occur upon, for example, exposure of the animal to a substance that promotes target gene alteration, introduction of an enzyme that promotes recombination at the target gene site (e.g., Cre in the Cre-lox system), or other method for directing the target gene alteration postnatally.

By “construct” is meant a recombinant nucleic acid sequence, generally recombinant DNA sequences, generated for the purpose of the expression of a specific nucleotide sequence(s), or is to be used in the construction of other recombinant nucleotide sequences.

By “operably linked” is meant that a DNA sequence and a regulatory sequence(s) are connected in such a way as to permit gene expression when the appropriate molecules (e.g., transcriptional activator proteins) are bound to the regulatory sequence(s).

By “operatively inserted” is meant that a nucleotide sequence of interest is positioned adjacent a nucleotide sequence that directs transcription and translation of the introduced nucleotide sequence of interest.

### DETAILED DESCRIPTION OF THE INVENTION

Methods of treating a host suffering from hyperlipidemia are provided. In the subject methods, an effective amount of an agent that at least reduces the plasma level of active apoE in the host, e.g. an apoE inhibitory agent, an agent that inhibits expression of apoE, etc., is administered to the host. The subject methods find particular use in the treatment of hyperlipidemias characterized by the presence of elevated levels of at least one of VLDL and triglycerides, e.g. Type IIb and Type IV hyperlipidemia. Also provided by the subject invention are non-human transgenic animal models of hyperlipidemia, where the subject animal models express high levels of human apoE, particularly apoE3. The subject animal models find use in various applications, including research applications to determine the role of apoE in lipid metabolism and screening applications to identity therapeutic agents for use in the treatment of hyperlipidemia. In further describing the subject invention, the subject methods will be described first, followed by a description of the subject transgenic animals and methods for using the subject animals, e.g. in screening assays.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

Methods of Reducing Plasma VLDL and/or Triglyceride Levels

As summarized above, the subject invention provides methods and compositions for at least reducing the plasma levels of VLDL and/or triglycerides in a host. By “at least reduce” is meant that the subject methods result in a reduction in the plasma level of VLDL and/or triglycerides of at least about 2 fold, usually at least about 3 fold and more usually at least about 4 fold, as compared to a control (i.e. untreated analogous host). For example, where the plasma level of VLDL in the host is elevated, ranging from about 60 to 100 mg/dl, the subject method results in a reduction of the plasma level of VLDL to a range of about 10 to 50 mg/dl, usually from about 15 to 30 mg/dl. Likewise, where the plasma level of triglycerides is elevated, ranging from about 300 to 600 mg/dl, the subject methods result in a reduction of plasma triglyceride to a level ranging from about 100 to 300 mg/dl, usually from about 100 to 200 mg/dl.

Critical to the subject methods is the administration of an agent to the host that at least reduces the plasma amount of active apolipoprotein E (apoE) in the host, including apoE2, apoE3 and apoE4, particularly apoE3. By active apoE is meant apoE that is able to function in its normal physiological role, e.g. mediation of lipoprotein uptake in the liver, and the like. Administration of the agent according to the subject methods results in at least a 2 fold reduction, usually at least about a 3 fold reduction and more usually at least about a 4 fold reduction in the plasma level of active apoE. For example, where the plasma level of apoE in the host is elevated, ranging from about 15 to 20 mg/dl, administration of the agent results in a reduction of plasma amount of active apoE to level ranging from about 3 to 6 mg/dl, usually from about 4 to 5 mg/dl.

The agent may reduce the amount of active plasma apoE in the host in a number of different ways. For example, the agent may be an apoE inhibitor, which agent interacts with plasma active apoE is such a manner as to render the apoE inactive, e.g. incapable of participating in its normal physiological roles, such as mediating lipoprotein clearance by LDL receptors, etc. The apoE inhibitor may be a number of different types of agents, such as small molecules, antibodies or binding fragments thereof, and the like.

Naturally occurring or synthetic small molecule compounds of interest include numerous chemical classes, though typically they are organic molecules, preferably small organic compounds having a molecular weight of more than 50 and less than about 2,500 daltons. Agents of interest typically comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

Also of interest as active agents are antibodies that at least reduce, if not inhibit, the apoE activity in the host. Suitable antibodies are obtained by immunizing a host animal with peptides comprising all or a portion of the apoE target protein. The protein sequences of the human apoE proteins are known, e.g. human apoE3 has a GENPEPT accession number of 1942471; apoE2 has a GENPEPT accession number of 1942472; and apoE precursor protein has a GENPEPT accession number of 114039. Purified apoE is also reported in: Barbier, et al., “Characterization of three human apolipoprotein E isoforms (E2, E3 and E4) expressed in Escherichia coli,” Eur. J. Clin. Chem. Clin. Biochem. (August 1997)35:581-9; Nukina et al., “Monoclonal antibody against the polymorphic site distinguishes apolipoprotein E4 from other isoforms,” Biochem. Biophys. Res. Commun. (Nov. 13, 1995) 216:467-72; and Pillot et al., “Single-step purification of two functional human apolipoprotein E variants hyperexpressed in Escherichia coli,” Protein Expr. Purif. (June 1996)7:407-14. Suitable host animals include mouse, rat sheep, goat, hamster, rabbit, etc. The origin of the protein immunogen may be mouse, human, rat, monkey etc. The host animal will generally be a different species than the immunogen, e.g. human apoE used to immunize mice, etc.

The immunogen may comprise the complete protein, or fragments and derivatives thereof. Preferred immunogens comprise all or a part of apoE, where these residues contain the post-translation modifications, such as glycosylation, found on the native target protein. Immunogens comprising the extracellular domain are produced in a variety of ways known in the art, e.g. expression of cloned genes using conventional recombinant methods, isolation from HEC, etc.

For preparation of polyclonal antibodies, the first step is immunization of the host animal with the target protein, where the target protein will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil & water emulsions, e.g. Freund's adjuvant, Freund's complete adjuvant, and the like. The target protein may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target protein is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using apoE bound to an insoluble support, protein A sepharose, etc.

The antibody may be produced as a single chain, instead of the normal multimeric structure. Single chain antibodies are described in Jost et al. (1994) J.B.C. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this fusion allows assembly of a functional variable region that retains the specificity and affinity of the original antibody.

For in vivo use, particularly for injection into humans, it is desirable to decrease the antigenicity of the antibody. An immune response of a recipient against the blocking agent will potentially decrease the period of time that the therapy is effective. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

Specific antibodies of interest include: those described Barbier, et al., “Characterization of three human apolipoprotein E isoforms (E2, E3 and E4) expressed in Escherichia coli,” Eur. J. Clin. Chem. Clin. Biochem. (August 1997)35:581-9; and Nukina et al., “Monoclonal antibody against the polymorphic site distinguishes apolipoprotein E4 from other isoforms,” Biochem. Biophys. Res. Commun. (Nov. 13, 1995) 216:467-72.

Also of interest are agents that inhibit the expression of apoE. For example, antisense molecules can be used to down-regulate the expression of the gene encoding apoE in cells of the host. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. The mRNA sequence of human apoE2 and apoE3 has a GENBANK accession number of K00396 and X00199, while the mRNA sequence of human apoE4 has a GENBANK accession number of M10065, J03053 and J03054. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

In practicing the subject methods, an effective amount of the agent is administered to the host to achieve the desired reduction in plasma VLDL and/or triglyceride levels in the host. By “effective amount” is meant a dosage sufficient to produce the desired amount of VLDL and/or triglyceride level reduction. Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means. For example, where the active agent is small molecule, the dosage may range from 1 ng to 1 g, usually from about 1 μg to 100 mg. Alternatively, where the active agent is an antibody composition, the dosage may range from about 1 μg to 1 g, usually from about 1 μg to 1 mg. In yet another embodiment in which the active agent is antisense, the dosage may range from about 1 ng to 1 mg, usually from about 1 μg to 100 μg.

In the subject methods, the active agent(s) may be administered to the host using any convenient means capable of resulting in the desired reduction in active apoE and concomitant reduction in serum VLDL and/or triglyceride levels. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

The subject methods may be used to reduce plasma VLDL and/or triglyceride levels in a variety of different hosts. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), lagomorph, e.g. rabbit, and primates (e.g., humans, chimpanzees, and monkeys). In many embodiments, the hosts will be humans.

The subject methods find use in the treatment of disease conditions associated with elevated lipid levels, i.e. hyperlipidemias. Of particular interest is the use of the subject methods to treat disease conditions associated with elevated plasma levels of VLDL and/or triglycerides. By elevated level of VLDL is meant a plasma VLDL level of at least about 20 mg/dl, usually at least about 30 mg/dl and more usually at least about 60 mg/dl, where the level may be as high as 100 mg/dl or higher. By elevated level of triglycerides is meant a total plasma triglyceride level (the total amount of all of the various types of triglycerides found in the plasma) of at least about 200 mg/dl, usually at least about 250 mg/dl and more usually at least about 300 mg/dl, where the level may be as high as 500 mg/dl or higher. Of particular interest is the use of the subject methods to treat apoE3 mediated hyperlipidemias, where specific conditions of interest include: Type IV hyperlipidemia or Type IIb hyperlipidemia, where such disease conditions are well known to those of skill in the art and described in Harrison's Principles of Internal Medicine (1998 ed), as well as the references cited therein.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as elevated plasma VLDL or triglyceride levels. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition, e.g. plasma VLDL and/or triglyceride levels are returned to normal.

Animal Models

The subject invention also provides non-human transgenic animal models of hyperlipidemia. By transgenic is meant an animal that comprises an exogenous nucleic acid sequence present as an extrachromosomal element or stably integrated in all or a portion of its cells, especially in germ cells. The exogenous nucleic acid sequence generally encodes human apoE, particularly human apoE3. A variety of different species of non-human animals are encompassed by the subject invention, where the subject animal model will typically be mammalian, including non-human primates, dogs, cats, cows, pigs, and the like, where species of the order rodentia, e.g. mice, rats and guinea pigs, as well as lagomorph, e.g. rabbits, are of particular interest. Preferably, the subject non-human animal model is a mouse or rabbit.

As the subject non-human animal models are animal models of human hyperlipidemia, they have plasma lipid levels which are analogous to human hyperlipidemia. Of particular interest are animals that have plasma lipid profiles that resemble human hypertriglyceridemia (HTG). In such animals, the plasma level of VLDL is at least about 10 mg/dl, usually at least about 15 mg/dl and more usually at least about 20 mg/dl, where the serum level may be as high as 60 mg/dl or higher. The plasma level of IDL ranges from about 10 to 50 mg/dl, and usually from about 15 to 30 mg/dl. The plasma level of HDL ranges from about 30 to 60 mg/dl and usually from about 40 to 50 mg/dl. The plasma level of triglycerides is at least about 100 mg/dl, usually at least about 150 mg/dl and more usually at least about 200 mg/dl, where the plasma level may be as high as 300 mg/dl or higher. The plasma level of cholesterol is at least about 120 mg/dl, usually at least about 150 mg/dl and more usually at least about 200 mg/dl, where the plasma level may be as high as 300 mg/dl or higher.

The plasma level of human apoE, particularly human apoE3, in the subject transgenic animals is sufficient to result in the above lipid profile. Generally, the plasma level of human apoE is at least about 30 mg/dl, usually at least about 35 mg/dl and more usually at least about 40 mg/dl, where the plasma level may be as high as 50 mg/dl or higher. Preferably, the subject transgenic animals do not express endogenous apoE, i.e. they are endogenous apoE knockout mice.

Where the subject transgenic animal model is a mouse, the animal has an apoE, preferably apoE3 plasma level of at least about 25 mg/dl, usually at least about 30 mg/dl and more usually at least about 35 mg/dl. The plasma lipid profile of the mouse is characterized as follows: VLDL ranges from about 10 to 60 mg/dl, usually from about 20 to 30 mg/dl; IDL ranges from about 10 to 50 mg/dl, usually from about 15 to 30 mg/dl; HDL ranges from about 30 to 60 mg/dl, usually from about 40 to 50 mg/dl; triglycerides range from about 100 to 500 mg/dl, usually from about 150 to 300 mg/dl; and cholesterol ranges from about 60 to 300 mg/dl, usually from about 100 to 250 mg/dl. In a preferred embodiment, the mouse is an LDL receptor knockout mouse, such that it does not express endogenous LDL receptors.

Where the subject transgenic animal model is a rabbit, the animal has an apoE3 plasma level of at least about 15 mg/dl, usually at least about 20 mg/dl and more usually at least about 25 mg/dl. The plasma lipid profile of the rabbit is characterized as follows: VLDL ranges from about 10 to 50 mg/dl, usually from about 20 to 40 mg/dl; IDL ranges from about 15 to 80 mg/dl, usually from about 30 to 60 mg/dl; HDL ranges from about 30 to 60 mg/dl, usually from about 40 to 50 mg/dl; triglycerides range from about 80 to 300 mg/dl, usually from about 100 to 200 mg/dl; and cholesterol ranges from about 80 to 350 mg/dl, usually from about 100 to 250 mg/dl.

The subject animals will generally comprise at least one human apoE transgene, e.g. a human apoE3 transgene, such that the animal will be a human apoE transgenic animal. The human apoE transgene carried by the animal will be one that is capable of being expressed in the animal in a manner sufficient to produce the above described lipid profile. Human apoE transgenes suitable for use in producing the subject animals are known in the art and/or readily obtained by those of skill in the art. See e.g. U.S. Pat. No. 5,767,337, the disclosure of which is herein incorporated by reference.

Methods for Producing the Subject Animal Models

The subject transgenic animal models can be produced as follows. DNA constructs comprising the human apoE gene for homologous recombination in embryonic stem (ES) cells are prepared, where such constructs may or may not comprise at least a portion of homology to a target locus, depending on whether site specific or random integration is desired. Conveniently, markers for positive and negative selection are included. Methods for generating cells having targeted gene modifications through homologous recombination are known in the art. For various techniques for transfecting mammalian cells, see Keown et al. (1990), Meth. Enzymol. 185:527-537. An ES cell line maybe employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). The resultant ES cells are then transformed with the human apoE DNA transgene construct, where transformation is accomplished using any convenient technique, e.g. electroporation, and the like. When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification are mated to produce homozygous progeny.

Methods of Using the Transgenic Animal Models

The subject transgenic animal models find use in a variety of applications, particularly in research applications, including research applications designed to elucidate the role of apoE in the development or progression of hyperlidemias, as well as in research applications designed to identify therapeutic agents for the treatment or amelioration of hyperlipidemias.

For example, as the subject animal models have lipid profiles analogous to those observed in human hyperlipidemic subjects, they can be used to study the effect of various genes and their expression products in the development of hyperlipidemias, particularly those characterized by elevated VLDL levels and/or triglyceride levels, including Type IIb and Type IV hyperlipidemias.

Of particular interest is the use of the subject animal models for the screening of potential hyperlipidemic therapeutic agents. Through use of the subject transgenic animals or cells derived therefrom, one can identify compounds that modulate the progression of hyperlidemias, e.g. by binding to, modulating, enhancing or repressing the activity of a protein or peptide involved in the progression of hyperlipidemia, e.g. apoE3. Screening to determine drugs that lack effect on the progression of hyperlipidemia is also of interest. Of particular interest are screening assays for agents that have a low toxicity for human cells. Assays of the invention make it possible to identify compounds which ultimately (1) have a positive affect with respect to hyperlipidemia and as such are therapeutics, e.g. agents which arrest or reverse the hyperlipidemia; or (2) have an adverse affect with respect to hyperlipidemia progression and as such should be avoided as therapeutic agents and in products consumed by animals, in particular humans.

A wide variety of assays may be used for this purpose, including lipid profile analysis studies, determination of the localization of drugs after administration, labeled in vitro protein-protein binding assays, protein-DNA binding assays, electrophoretic mobility shift assays, immunoassays for protein binding, and the like. Depending on the particular assay, whole animals may be used, or cells derived therefrom. Cells may be freshly isolated from an animal, or may be immortalized in culture.

The term “agent” as used herein describes any molecule, e.g. protein or non-protein organic pharmaceutical, with the capability of affecting any of the biological actions underlying hyperlipidemia. Generally a plurality of assay mixtures are run in parallel with different agent concentrations to obtain a differential response to the various concentrations. Typically, one of these concentrations serves as a negative control, i.e. at zero concentration or below the level of detection. Candidate agents include those agents described above in connection with the description of the methods of reducing VLDL and/or triglyceride levels in a host. As such, screening may be directed to known pharmacologically active compounds and chemical analogs thereof, or to new agents with unknown properties such as those created through rational drug design. Efficacious candidates can be identified by phenotype, i.e. return to normal lipid profile, return to normal apoE level, and the like.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Samples, as used herein, include biological fluids such as tracheal lavage, blood, cerebrospinal fluid, tears, saliva, lymph, dialysis fluid and the like; organ or tissue culture derived fluids; and fluids extracted from physiological tissues. Also included in the term are derivatives and fractions of such fluids. The number of cells in a sample will generally be at least about 10³, usually at least 10⁴ more usually at least about 10⁵. The cells may be dissociated, in the case of solid tissues, or tissue sections may be analyzed. Alternatively a lysate of the cells may be prepared.

For example, detection may utilize staining of cells or histological sections, performed in accordance with conventional methods. The antibodies of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method depends on the in vitro detection of binding between antibodies and a protein of interest in a lysate. Measuring the concentration of binding in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Alternatively, lipid profiles of the transgenic animals can be determined using methods well known to those of skill in the art, where such methods are described in Shimano et al., P.N.A.S. USA (March 1992) 89: 1750-1754; Fan et al., J. Clinical Invest. (May 1998) 10:2151-2164; Huang et al., J. Biol. Chem. (Oct. 9, 1998) 273:26388-26393; Huang et al., J. Biol. Chem. (Jul. 10, 1998) 273:17483-17490; and Sullivan et al., J. Biol. Chem. (Jul. 18, 1997) 272:17972-17980; as well as in U.S. Pat. No. 5,767,337 and WO 97/05247, the disclosures of which are herein incorporated by reference.

The following examples are offered by way of illustration and not by way of limitation.

### EXPERIMENTAL

I. Mouse Studies

A. Materials and Methods

Materials

A Superose 6 column purchased from Pharmacia was used on a Pharmacia fast protein liquid chromatography system. Cholesterol and triglyceride standards were from Abbott (North Chicago, Ill.) and Boehringer Mannheim (Mannheim, Germany), respectively. The automated system for lipid analysis (Kinetic Microplate Reader) was from Molecular Devices (Menlo Park, Calif.). Triton WR1339, oleic acid, free fatty acid-free bovine serum albumin, and bovine milk lipoprotein lipase (LPL) were from Sigma. [¹⁴C]acetate, and ECL chemiluminescence detection kits for western blots were purchased from Amersham Life Science (Little Chalfont, Buckinghamshire, United Kingdom.

Transgenic Mice

Hemizygous human apoE3 transgenic mice (ICR strain) were produced at the Gladstone Institute of Cardiovascular Disease with a DNA construct containing human apoE3 genomic DNA and the hepatic control region (Simonet, W. S., Bucay, N., Lauer, S. J., and Taylor, J. M. (1993) J. Biol. Chem. 268, 8221-8229). Homozygous apoE knockout (mE0) and homozygous LDL receptor knockout (LDLR0) mice (C57BL/6 strain) were purchased from Jackson Laboratories (Bar Harbor, Me.). ApoE and LDL receptor double-knockout mice (mE0/LDLR0) were generated in our laboratory by crossbreeding apoE knockout mice with LDL receptor knockout mice.

Male transgenic mice expressing low (apoE3=8 mg/dl; E3 low) or high (apoE3=22 mg/dl; E3 high) levels of human apoE3 were crossbred with female apoE knockout mice (mE0) to generate apoE3 transgenic mice without endogenous mouse apoE (E3low/mE0 and E3high/mE0). The human apoE3 transgene was detected by immunoblotting plasma (1 μl) with human-specific anti-apoE polyclonal antiserum (Huang et al., J. Biol. Chem. (1997) 271:29146-29151. Mouse apoE deficiency was established by western blotting with mouse-specific anti-apoE antiserum (provided by Dr. Jan Borén, Gladstone Institute of Cardiovascular Disease). In some cases, E3low/mE0 or E3high/mE0 mice were crossbred with LDL receptor-null mice lacking mouse apoE (mE0/LDLR0) to generate E3low/mE0 or E3high/mE0 mice on a heterozygous (E3low/mE0/LDLR1 or E3high/mE0/LDLR1) or a homozygous (E3low/mE0/LDLR0 or E3high/mE0/LDLR0) LDL receptor-null background. LDL receptor deficiency was assessed by polymerase chain reaction with specific primers designed to identify both the altered and the unaltered gene sequences (Huang et al., Aterioscler. Thromb. Vasc. Biol. (1997) 17:2817-2824.

Human Subjects

Human subjects were selected from the PROCAM Study group (Assmann et al. Am. J. Cardiol. (1996)77:1179-1184) at the Arteriosclerosis Research Institute, University of Müster, Münster, Germany. Type IV hyperlipidemic subjects (n=27) were defined as having plasma triglycerides >200 mg/dl and LDL cholesterol <140 mg/dl (18). Normolipidemic subjects (n=6) were defined as having plasma triglycerides <200 mg/dl and LDL cholesterol <140 mg/dl.

Lipoprotein Separation and Analysis

Mouse blood was collected from the tails of 8-16-week-old mice that had been fasted for 5 h. Human blood was collected from normolipidemic and type IV hyperlipidemic subjects after an overnight fast; EDTA was used as an anti-coagulant. Plasma was obtained by centrifugation at 14,000 rpm (microfuge) for 10 min (mouse samples) or at 3000 rpm for 15 min (human samples) at 4° C. Cholesterol and triglyceride levels were measured on total plasma and on chromatographic fractions by an enzymatic colorimetric method adapted for use with a microplate reader (19, 20).

Lipoproteins of mouse or human plasma (100 μl) were separated by chromatography on a Superose 6 column as described previously (Huang (1996) supra). The major lipoprotein classes eluted from the column were pooled and concentrated with Centricon filters (fractions 16-18, VLDL; fractions 19-22, intermediate density lipoprotein; fractions 23-27, LDL and HDL₁; and fractions 28-33, HDL). To analyze the distribution of apoE in various lipoproteins, the pooled samples representing different lipoprotein classes were separated on a 12% polyacrylamide-SDS gel followed by immunoblotting with anti-human apoE antiserum. For analysis of chemical compositions and lipolysis of VLDL, the VLDL (d<1.006 g/ml) were isolated from both mouse and human plasma by ultracentrifugation at 100,000 rpm for 2 h at 4° C. in a Beckman TL100 ultracentrifuge (de Silva et al., J. Lipid Res. (1994) 35:1297-1310). Cholesterol and triglyceride levels were measured as described above. Apolipoproteins were separated on 10-20% polyacrylamide-SDS gradient gels. The amounts of human or mouse apoE and human or mouse apoC-II were determined by western blotting with polyclonal antibodies against human apoE, mouse apoE, human apoC-II, and mouse apoC-II, respectively (kindly provided by Dr. Karl Weisgraber, Gladstone Institute of Cardiovascular Disease) (Huang et al., (1997) supra, Huang et al., J. Biol. Chem (1998) 273:17483-17490; and Weisgraber et al., J. Biol. Chem. (1990) 265:22453-22459. Purified human apoE, mouse apoE, human apoC-II, and mouse apoC-II were used as standards, respectively (provided by Dr. Karl Weisgraber).

VLDL Triglyceride Production in Vivo

Hepatic VLDL triglyceride production was determined with the Triton WR1339 method (Aalto-Setala et al., J. Clin. Invest. (1992)) 90:1889-1900; and Kuipers et al., J. Clin. Invest. (1997) 100:2915-2922. Briefly, nontransgenic or various apoE3 transgenic mice were injected intravenously with 500 mg of Triton WR1339 (300 mg/ml in 0.9% NaCl) per kg body weight after an overnight fast. Blood samples (50 μl) were collected 0, 15, 30, 60, and 90 min later. Plasma triglyceride concentrations were measured as described above. The hepatic VLDL triglyceride production rate was calculated from the slope of the curve and presented as μM/h/kg.

VLDL Triglyceride Production in Vitro in ApoE-transfected Rat Hepatoma Cells

To determine the effects of apoE expression levels on VLDL triglyceride production in vitro, rat hepatoma cells (McA-RH7777) were cotransfected with various apoE isoform genomic DNA and a neomycin gene Ji et al., J. Biol. Chem. (1994) 269: 2764-2772. Stably transfected colonies were selected by culturing the cells in medium containing G418 (400 μg/ml) for 2 weeks Positive colonies were characterized by reverse transcriptase-polymerase chain reaction with primers specific for the human apoE gene and by anti-human apoE immunoblotting. Over 140 colonies were screened, and three transfected cell lines for each apoE isoform, which had matched apoE secretion levels, were selected for study.

VLDL triglyceride production rates were determined by incubating nontransfected and transfected cells with Dulbecco's modified Eagle's medium containing 1% bovine serum albumin, 1 mM oleic acid, and [¹⁴C]acetate (5 μCi/ml) at 37° C. for 4 h (26) in the absence or presence of 12 units/ml of heparinase (Ji et al., J. Biol. Chem. (1993) 268:10160-10167. After incubation, the media were collected, and VLDL isolated by ultracentrifugation at d<1.006 g/ml. Lipids were extracted from VLDL with chloroform/methanol (2:1), separated by thin-layer chromatography, and quantitated by measuring the radioactivity of each fraction.

Lipolysis of VLDL in Vitro

To determine the ability of normal and apoE-enriched VLDL to serve as substrates for lipase-mediated lipolysis, various VLDL samples (30 μg of triglycerides) were incubated with 1 μg of bovine milk LPL or 50 μl of hepatic lipase-transfected cell-conditioned medium for 30 min at 37° C. In some cases, specific amounts of purified human apoE3 or apoC-II were included in the incubation (Huang et al., J. Biol. Chem. (1998) 273:17483-17490. After incubation, the levels of released free fatty acids were determined by an enzymatic colorimetric method Connelly et al., J. Biol. Chem. (1994) 269:20554-20560. (Wako Chemicals, Richmond, Va.).

B. Results

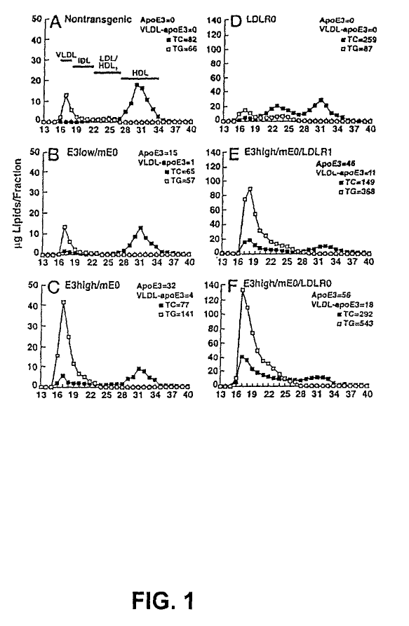

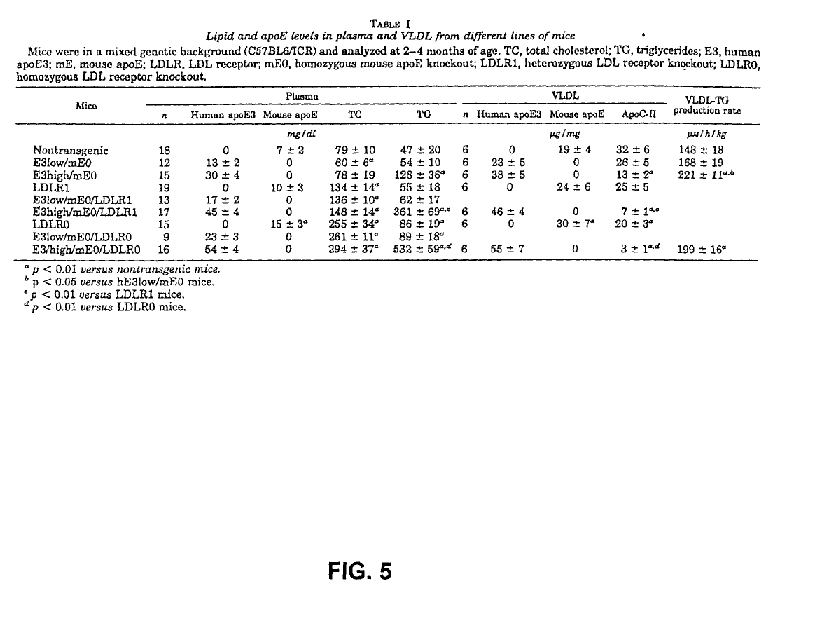

In studying the effects of apoE on triglyceride-rich lipoprotein metabolism, it was found that hepatic overexpression of human apoE3 at high levels (˜30 mg/dl) in transgenic mice lacking endogenous mouse apoE (E3high/mE0) led to mild HTG (an ˜3-fold increase in plasma triglycerides versus nontransgenic mice) without significant changes in plasma total cholesterol (Table I, FIG. 5). In contrast, low levels of apoE3 expression (˜13 mg/dl) on the same genetic background (E3low/mE0) did not alter plasma triglyceride levels significantly (Table I, FIG. 5; FIG. 1B). The E 3high/mE0 mice had increased VLDL triglyceride and cholesterol and decreased HDL cholesterol (FIG. 1C versus 1A), with accumulation of apoE3 in the VLDL fraction (Table I, FIG. 5). Thus, overexpression of apoE3 in transgenic mice alters the plasma lipoprotein profile to one that resembles the human HTG phenotype.

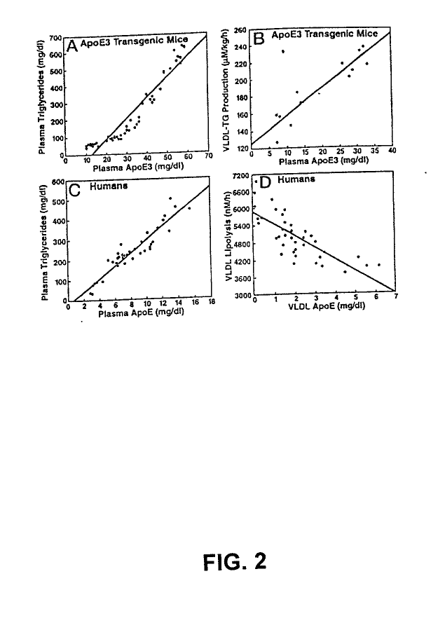

To determine if further accumulation of apoE3 in mouse plasma would exacerbate the HTG, we crossed the E3high/mE0 mice with LDL receptor knockout mice (LDLR0) to eliminate one of the pathways for apoE clearance. Removing one (E3high/mE0/LDLR1) and then both (E3high/mE0/LDLR0) LDL receptor alleles increased plasma apoE3 by 50% and 80% (Table I, FIG. 5) and plasma triglyceride levels by ˜3-and ˜4-fold, respectively, compared with the E3high/mE0 mice. VLDL cholesterol and triglycerides also increased significantly (compare FIG. 1C with FIGS. 1, E and F). Thus, additional accumulation of apoE3 caused by the LDL receptor deficiency further exacerbates the apoE3 overexpression-induced HTG phenotype. Interestingly, removal of LDL receptors from the E3low/mE0 mice did not significantly alter plasma triglyceride levels, possibly because the apoE3 levels were not elevated sufficiently to affect triglyceride levels (Table I, FIG. 5). Thus, rather than an absence of LDL receptors, an increased apoE3 level seems to be an important determinant of plasma triglyceride metabolism. In fact, plasma triglycerides correlated positively with apoE levels in apoE3 transgenic mice (FIG. 2A). Triglyceride levels increased ˜11-fold as plasma apoE3 rose from ˜10 to ˜55 mg/dl (FIG. 2A).

At least two mechanisms could explain the HTG associated with apoE3 overexpression: stimulated VLDL triglyceride production and impaired VLDL lipolysis. To ascertain if apoE3-overexpressing mice had increased VLDL triglyceride production, a characteristic of human HTG, we determined in vivo VLDL triglyceride production rates in various apoE3 transgenic mouse lines using intravenous administration of Triton WR1339 to inhibit lipolysis. The E3high/mE0 mice had a 50% increase in VLDL triglyceride production rate compared with nontransgenic mice, whereas the E3low/mE0 mice had no significant change (Table I, FIG. 5). The VLDL triglyceride production rate correlated positively with plasma apoE levels (FIG. 2B), suggesting that apoE3 overexpression-induced HTG is at least partially due to stimulation of VLDL triglyceride production.

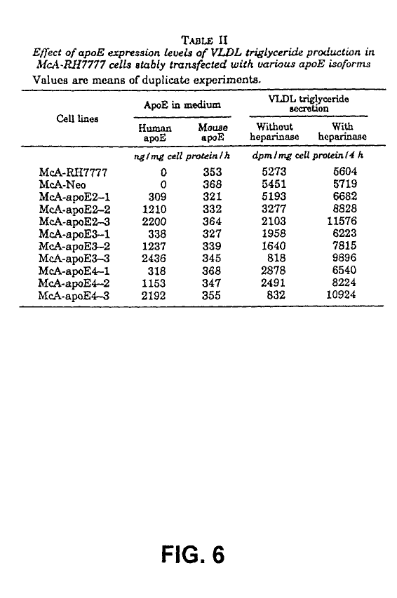

The effect of apoE on VLDL synthesis and/or secretion was further established by expressing different levels of apoE2, E3, or E4 in rat hepatoma cells (McA-RH7777). Three transfected cell lines were selected for each apoE isoform according to their expression levels of apoE, which varied over 7-fold from lowest to highest (Table II, FIG. 6). Interestingly, expression levels of endogenous rat apoE were not changed by overexpressing human apoE. Increasing levels of apoE expression actually resulted in decreased VLDL triglyceride secretion into the medium. However, an increase in apoE likely stimulates the reuptake of secreted VLDL via the heparan sulfate proteoglycan/LDL receptor-related protein pathway. Treatment of the transfected cells with heparinase to block this pathway clearly resulted in a dose-dependent increase in VLDL triglyceride secretion (Table II, FIG. 6). Thus, an increase in apoE expression by the hepatoma cells correlates with increased VLDL synthesis and/or secretion. Similar results were obtained with all three apoE isoforms, confirming that the effect is independent of isoform type.

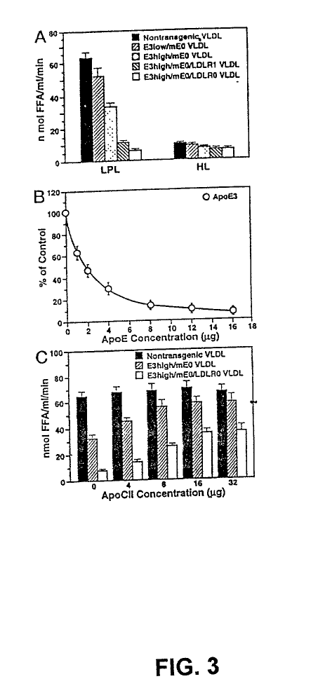

However, the VLDL triglyceride production rate in the E3high/mE0/LDLR0 mice did not differ significantly from that of the E3high/mE0 mice, even though the former had 4-fold higher plasma triglyceride levels than the latter (Table I, FIG. 1). Thus, VLDL triglyceride overproduction is only one aspect of the mechanism(s) responsible for the severe HTG in E3high/mE0/LDLR0 mice. The increase in plasma triglycerides and VLDL cholesterol with the simultaneous decrease in LDL and HDL cholesterol in the presence of increasing levels of apoE3 in the E3high/mE0/LDLR1 and E3high/mE0/LDLR0 mice raised the possibility that the accumulation of apoE3 impaired the lipolytic conversion of VLDL to LDL, as previously suggested for apoE2. To test this hypothesis, we examined the abilities of normal and transgenic VLDL containing various amounts of apoE3 (Table I, FIG. 5) to serve as substrates for lipase-mediated lipolysis in vitro (FIG. 3A). Accumulation of apoE3 in transgenic VLDL from E3high/mE0, E3high/mE0/LDLR1, and E3high/mE0/LDLR0 mice inhibited LPL-mediated lipolysis by 48%, 83%, and 86%, respectively, compared with the VLDL from nontransgenic mice. Hepatic lipase-mediated lipolysis was affected to a lesser degree. Thus, apoE3 -enriched VLDL, like apoE2-enriched VLDL, are poorer substrates for LPL-mediated lipolysis than normal VLDL. The inhibitory effect of apoE3 on lipolysis was confirmed by adding increasing amounts of purified human apoE3 to nontransgenic VLDL (FIG. 3B). More than 90% of LPL-mediated lipolysis was inhibited at the highest apoE3 levels.

To explain the inhibitory effect of apoE3 on lipolysis, we found that apoC-II content in the transgenic VLDL decreased gradually with increasing amounts of apoE3 (Table I, FIG. 5), suggesting that apoE3 accumulation in the VLDL may displace apoC-II, a well-defined cofactor for LPL activity. To address this possibility, we added purified human apoC-II to various VLDL and determined its effects on lipolysis (FIG. 3C). Adding apoC-II to apoE3-enriched VLDL stimulated LPL-mediated lipolysis in a dose-dependent manner, indicating that apoE3-impaired lipolysis of VLDL can be at least partially corrected by increasing the amount of apoC-II on the particles.

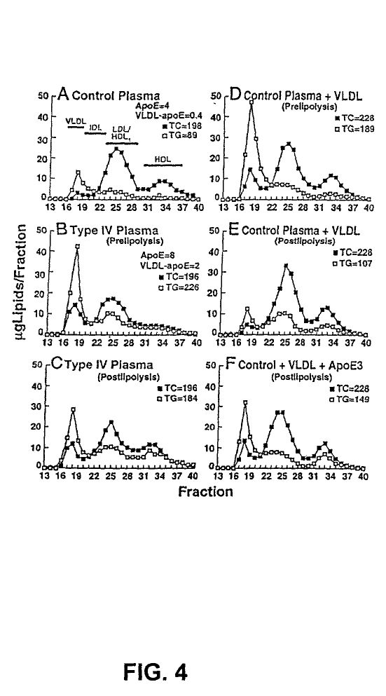

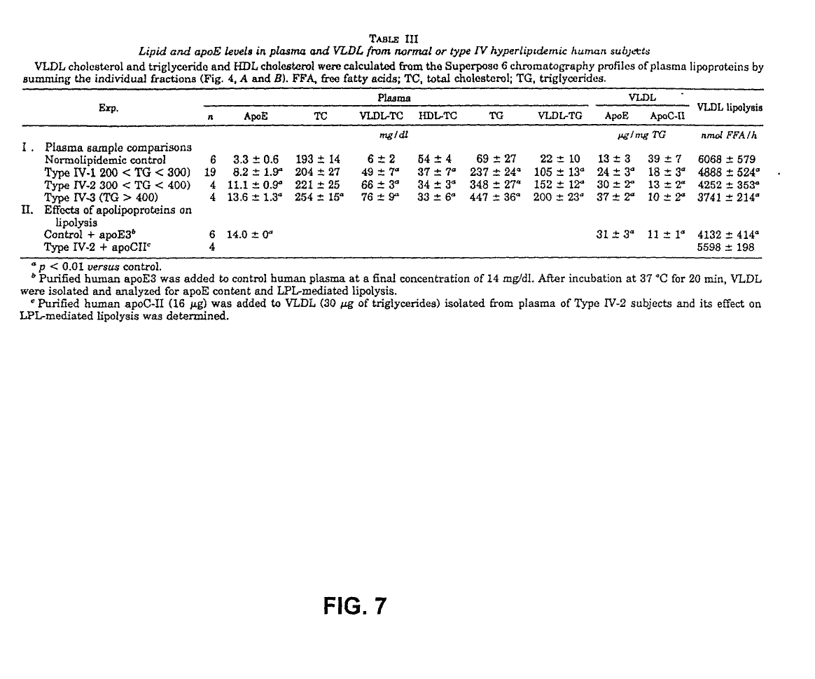

To test apoE involvement in the development of HTG in humans, we examined plasma samples from 27 patients with HTG and six normal controls (Table III, FIG. 7). Consistent with previous reports, the HTG subjects had increased plasma triglycerides, increased VLDL cholesterol and triglycerides, and decreased HDL cholesterol (Table III, FIG. 7; compare FIGS. 4, A and B). Plasma apoE levels were 2.5- to 4-fold higher in the HTG patients than in normal controls, suggesting that overexpression and/or accumulation of apoE occurs in the HTG patients. As in apoE3 transgenic mice, plasma triglyceride levels and plasma or VLDL apoE levels were positively correlated (FIG. 2C) as were increased plasma triglyceride levels and VLDL apoE (r=0.94,p<0.001). Furthermore, plasma or VLDL apoE correlated negatively with VLDL lipolysis (FIG. 2D) and HDL cholesterol levels (r=0.51,p<0.001 and r=−0.55,p<0.001, respectively) (Table III). These data indicate that accumulation of apoE in the VLDL of HTG patients also impairs LPL-mediated lipolysis.

To confirm that the increase in apoE impairs LPL-mediated lipolysis, we compared the lipolytic conversion of VLDL to LDL in HTG plasma with that in normal plasma. Autologous VLDL were added to normal plasma to increase VLDL triglyceride to the level in the HTG plasma (compare FIG. 4, B and D). We then added 5 μg of LPL to each of the samples and incubated them at 37° C. for 1 h. As shown in FIG. 3C versus 3E, the VLDL in the HTG plasma were resistant to lipolytic processing compared with VLDL in normal plasma.

To determine whether the increased plasma apoE levels cause or result from increased triglycerides, purified human apoE3 was added to normal control plasma to levels similar to those in the HTG patients (14 mg/dl), and the effects of the increased apoE3 on VLDL lipolysis were determined. The increased apoE3 in the VLDL (2.5-fold) resulted in a 3-fold reduction in the apoC-II content of the VLDL and a marked impairment of LPL-mediated lipolysis of the VLDL (Table III, FIG. 7). Furthermore, apoE3 added to control plasma containing autologous VLDL resulted in an impaired lipolytic processing of VLDL to LDL (compare FIG. 4, E and F). In contrast, adding apoC-II to the VLDL from HTG patients (Type IV−2+apoC-II, Table III, FIG. 7) returned lipolysis to nearly normal levels. Taken together, these results indicate that HTG patients have a disturbance in lipoprotein metabolism similar to that of transgenic mice overexpressing apoE3 on either the mE0 or LDLR0 background [i.e., increased plasma and VLDL apoE, elevated plasma triglycerides and VLDL, decreased HDL, impaired VLDL lipolysis, and probably also increased VLDL triglyceride production].

II. Rabbit Studies

A. Materials and Methods

New Zealand White rabbits were purchased from Charles River. A Superose 6 column, purchased from Pharmacia, was used on a Pharmacia fast protein liquid chromatography system. Centricon concentration filters were from Amicon. Cholesterol standard was from Abbott. Triglyceride standard and assay kits were from Boehringer Maniheim. An automated system (Kinetic Microplate Reader) was used for lipid analysis. Triton WRI339, oleic acid, bovine serum albumin without free fatty acids, bovine milk lipoprotein lipase (LPL), and heparinase I were from Sigma. The ECL chemiluminescence detection kit for western blots was from Amersham Life Science.

Transgenic Rabbits.

Transgenic rabbits expressing different plasma levels of human apoE3 were generated previously at the Gladstone Institute of Cardiovascular Disease with a DNA construct containing the human apoE3 gene and its hepatic control region (Fan et al., J. Clin. Invest. (1998) 101:2151-2164). Transgene expression was detected by immunoblotting rabbit plasma (1 μL) with human-specific anti-apoE antiserum (Fan et al., supra; Huang et al., (1997) supra). In the western blot assay, human apoE3 was semiquantitated by comparing the densitometric readings of the sample bands with those of different concentrations of purified human apoE. Antibodies and apoE standards were provided by K. H. Weisgraber (Gladstone Institute of Cardiovascular Disease, San Francisco, Calif.). All experiments were performed under protocols approved by the Committee on Animal Research, University of California, San Francisco.

Lipoprotein Separation and Analysis.

Blood was collected from the intermedial auricular artery of 8-12-month-old rabbits that had been fasted overnight. EDTA was used as anticoagulant (final concentration, 10 mM). Plasma was obtained by centrifugation at 14,000 rpm (microcentrifuge) for 10 minutes at 4° C., and samples were stored for no more than 2 days at 4° C. in the presence of 1 mM phenylmethylsulfonyl fluoride, a protease inhibitor.

Lipoproteins in 200 μL of plasma were separated by chromatography on a Superose 6 column, as described previously (Huang et al., J. Biol. Chem. (1997) supra; Huang et al., (1996) supra). The major lipoprotein classes eluted from the column were pooled and concentrated with Centricon filters [fractions 16-18, VLDL; fractions 19-22, intermediate density lipoproteins (IDL); fractions 23-27, LDL and a subclass of high density lipoproteins (HDL₁); and fractions 28-33, high density lipoproteins]. Cholesterol and triglycerides were measured on total plasma and on chromatographic fractions by an enzymatic colorimetric method adapted for use with a microplate reader (Huang et al., (1997) supra; Huang et al., Arteroscler. Thromb. Vasc. Biol (1997) supra). Cholesterol and triglycerides in VLDL, IDL, and LDL were calculated from the Superose 6 chromatographic profiles of plasma lipoproteins by summing the individual fractions.

For analysis of apolipoprotein composition or lipolysis assays, VLDL (d<1.006 g/mL), IDL (d=1.006-1.02 g/mL), and LDL (d=1.02-1.06 g/mL) were isolated from rabbit plasma by ultracentrifugation at 100,000 rpm for 2 hours at 4° C. in a Beckman TL100 ultracentrifuge (de Silva (1994) supra). Cholesterol and triglyceride levels were measured as described above. Apolipoproteins were separated on 3-20% polyacrylamide-SDS gradient gels and detected by Coomassie blue staining. There was no detectable apoB48 in the d<1.006 g/mL fractions.

VLDL Triglyceride Production in Vivo.

Hepatic VLDL triglyceride production was determined with the Triton WR1339 method (Aalto-Setala et al., (1992) supra; Kuipers et al., (1997) supra). Briefly, nontransgenic or apoE3 transgenic rabbits were injected intravenously with 500 mg of Triton WR1339 (400 mg/mL in 0.9% NaCl) per kg of body weight after an overnight fast. Blood samples (1 mL) were collected 0, 15, 30, 60, and 90 minutes later. Plasma triglyceride concentrations were measured as described above. The hepatic VLDL triglyceride production rate was calculated from the slope of the curve and presented as μmol/kg/h.

Lipolysis of VLDL and IDL in Vitro.

The susceptibility of VLDL (d<1.006 g/mL) and IDL (d=1.006-1.02 g/mL) to lipolysis was determined by incubating 30 μg of lipoprotein triglycerides with 1 μg of LPL in PBS (pH 7.4) without serum for 30 minutes at 37° C. The levels of released free fatty acids were determined before and after incubation by an enzymatic colorimetric method (Connelly et al., (1994) supra) (Wako Pure Chemical Industries). Lipolysis was calculated by subtracting the values before incubation from the values after incubation. As reported previously (Bilheimer et al., Biochim. Biophys. Acta (1972) 260: 212-221), the intra- and inter-assay coefficients of variation for this assay were ˜7 and ˜9%, respectively.

VLDL Clearance.

The VLDL (d<1.006 g/mL) isolated from plasma of 4-5 rabbits from each of the nontransgenic and apoE3 transgenic groups were pooled and iodinated by the method of Bilheimer et al. The ¹²⁵I-labeled VLDL (5 μg of protein in 100 μl of saline) were injected into the tail vein of normal mice. At each time interval (0, 5, 10, and 20 minutes), three mice were euthanized, blood was collected via heart puncture, and the liver was removed. The removal of ¹²⁵I-VLDL from plasma was determined as described previously (Ji et al., (1995) supra; Ji et al., (1994) supra). A liver sample was taken for quantitation of uptake of the ¹²⁵I-VLDL. Plasma clearance and liver uptake were calculated on the basis of the percent of the injected dose of labeled material at different time points after injection. A plasma volume of 3.5% of body weight was used for the calculation.

Cell Association of VLDL.

Cultured HepG2 cells were grown to ˜100% confluence, washed three times with fresh serum-free medium, and incubated at 37° C. with ¹²⁵I-VLDL (5 μg of protein). In some cases, the cells were pretreated at 37° C. with heparinase I (10 units/mL) for 1 hour. The cells were then incubated in the presence of the heparinase with ¹²⁵I-VLDL for 2 hours and washed five times on ice with 0.1 M phosphate-buffered saline containing 0.2% bovine serum albumin and once with 0.1 M phosphate-buffered saline. The cell-associated radioactivity (from both cell-surface bound and internalized lipoproteins) was then counted, as described previously (Huang et al., J. Biol. Chem. (1998) 273:17483-17490).

Statistical Analysis.

Mean lipid levels are reported as the mean ±SD. Differences in lipids, apolipoproteins, or VLDL triglyceride production were evaluated by the t test. Correlation of plasma apoE3 with VLDL triglyceride production or VLDL and IDL lipolysis was assessed by regression analysis.

B. Results

Effects of ApoE3 Overexpression on Plasma Lipids and Lipoproteins.

The offspring (F1 hemizygotes) of two previously generated transgenic rabbit lines that expressed low (<10 mg/dL) or medium (10-20 mg/dL) levels of plasma human apoE3, respectively, were used in this study. To generate a high-expresser line (>20 mg/dL), F2 homozygous transgenic rabbits were established from the medium-expresser line. As reported previously, overexpression of the human apoE transgene in rabbits did not significantly alter endogenous rabbit apoE gene expression (data not shown).

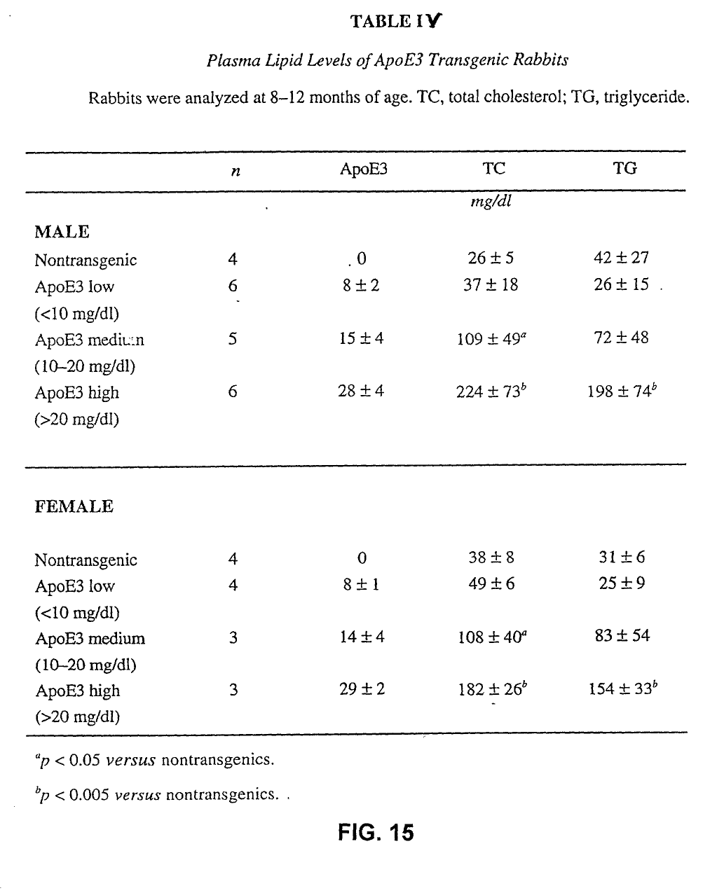

Table IV (FIG. 15) summarizes the plasma lipid levels in various apoE3 transgenic rabbit lines and nontransgenic rabbits at 8-12 months of age. In transgenic males and females, plasma total cholesterol levels were 3-4-fold higher in medium expressers (10-20 mg/dL) and 5-9-fold higher in high expressers (>20 mg/dL) than in nontransgenic rabbits. However, plasma triglyceride levels showed little change in low and medium expressers, but were markedly increased in high expressers (5-fold). Since no significant gender difference was observed (Table IV, FIG. 15), male rabbits were used for subsequent studies.

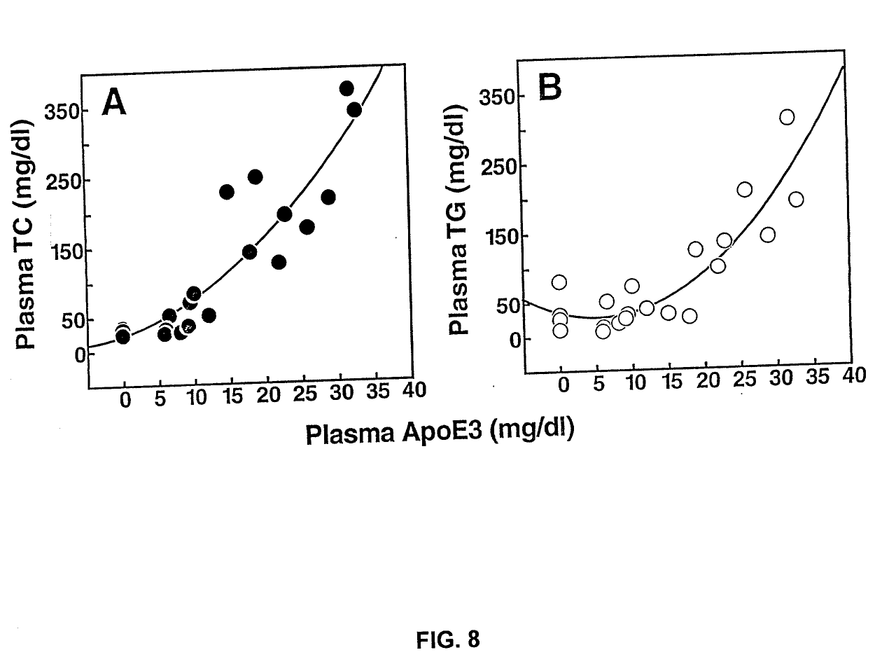

The correlation between plasma lipid and apoE3 levels for the 21 male rabbits indicated in Table IV (FIG. 15) is shown in FIG. 8. Plasma total cholesterol increased proportionally with increasing levels of apoE3, whereas plasma triglycerides remained unchanged (or slightly decreased) at apoE levels <20 mg/dL but increased sharply at higher levels (>20 mg/dL). Thus, apoE3 overexpression differentially affects plasma cholesterol and triglyceride levels, leading to hypercholesterolemia in the medium expressers and to combined hyperlipidemia in the high expressers.

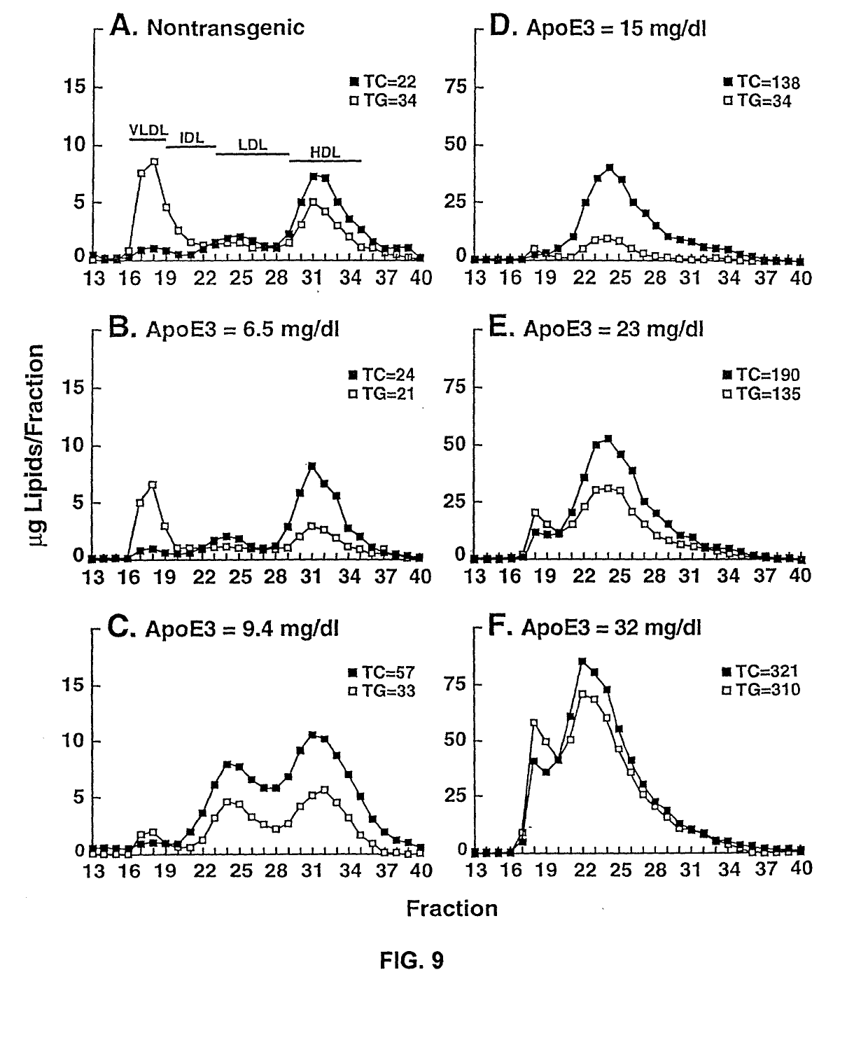

The changes in specific lipoproteins in response to apoE3 expression levels, as analyzed by gel filtration chromatography on a Superose 6 column, are shown in FIG. 9. A typical transgenic rabbit expressing a low amount of human apoE3 (6.5 mg/dL) (FIG. 9B) had a lipoprotein profile that was not significantly different from that in nontransgenic rabbits (FIG. 9A). However, a transgenic rabbit with an apoE3 concentration of 9.4 mg/dL had significantly higher LDL cholesterol and lower VLDL triglyceride (FIG. 9C). At an apoE3 level of 15 mg/dL (FIG. 9D), LDL cholesterol was dramatically increased, and VLDL cholesterol and triglyceride were further decreased. At apoE3 levels >20 mg/dL (FIGS. 9E and 9F), VLDL and IDL cholesterol and triglyceride levels were significantly increased, and LDL cholesterol remained at high levels.

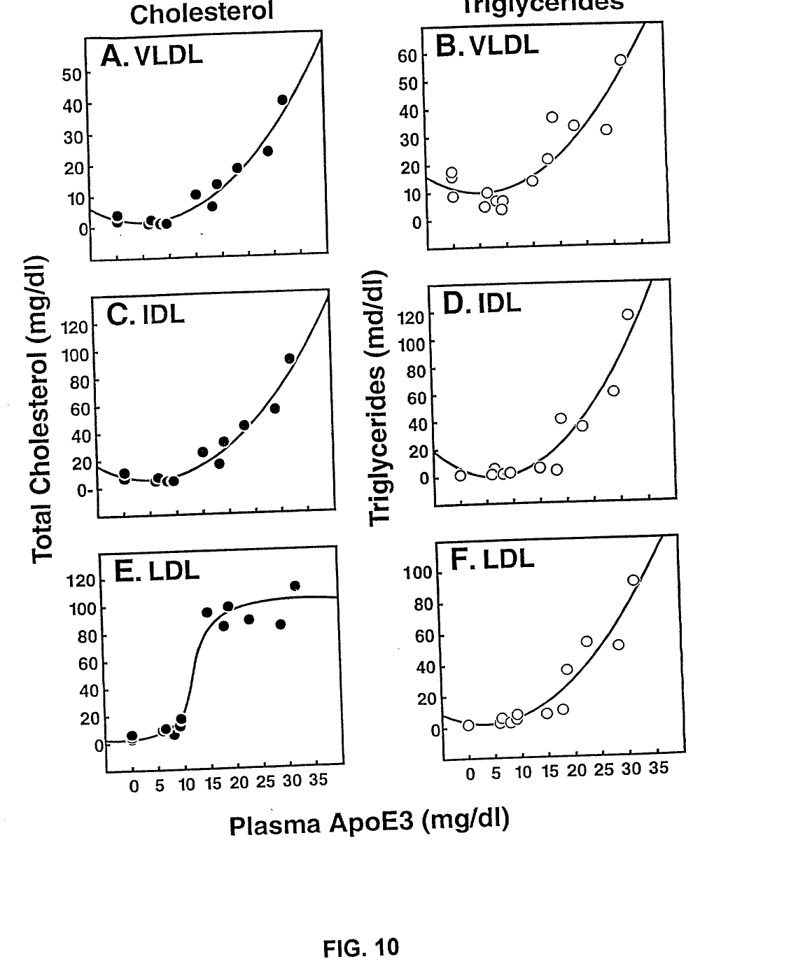

Changes in the cholesterol and triglyceride content of the various lipoproteins as a consequence of increased apoE3 expression are shown in FIG. 10. With the elevation of plasma apoE3 levels from 10 to 20 mg/dL, there was a nearly step-wise increase in LDL cholesterol (˜18-fold over nontransgenic controls) (FIG. 10E), only slight increases in VLDL and IDL cholesterol (FIGS. 10A and 10C), and no significant changes in VLDL and IDL triglycerides (FIGS. 10B and 10D). Thus, the hypercholesterolemia associated with medium levels of apoE3 overexpression (Table IV (FIG. 15) and FIG. 8) is due to a dramatic accumulation of LDL cholesterol. In contrast, at plasma apoE3 levels >20 mg/dL, there was no further change in LDL cholesterol (˜19-fold over nontransgenic controls) (FIG. 10E), whereas VLDL and IDL cholesterol and triglycerides increased progressively with increasing levels of plasma apoE3 (FIGS. 10A through 10D), as did LDL triglycerides (FIG. 10F). These results indicate that the hypercholesterolemia associated with medium levels of apoE3 overexpression (10-20 mg/dL) is due mainly to the accumulation of cholesterol-rich LDL, whereas the combined hyperlipidemia associated with high levels of apoE3 overexpression (>20 mg/dL) is due to the accumulation of cholesterol and triglycerides in VLDL and IDL.

Effects of ApoE3 Overexpression on Hepatic VLDL Triglyceride Production.

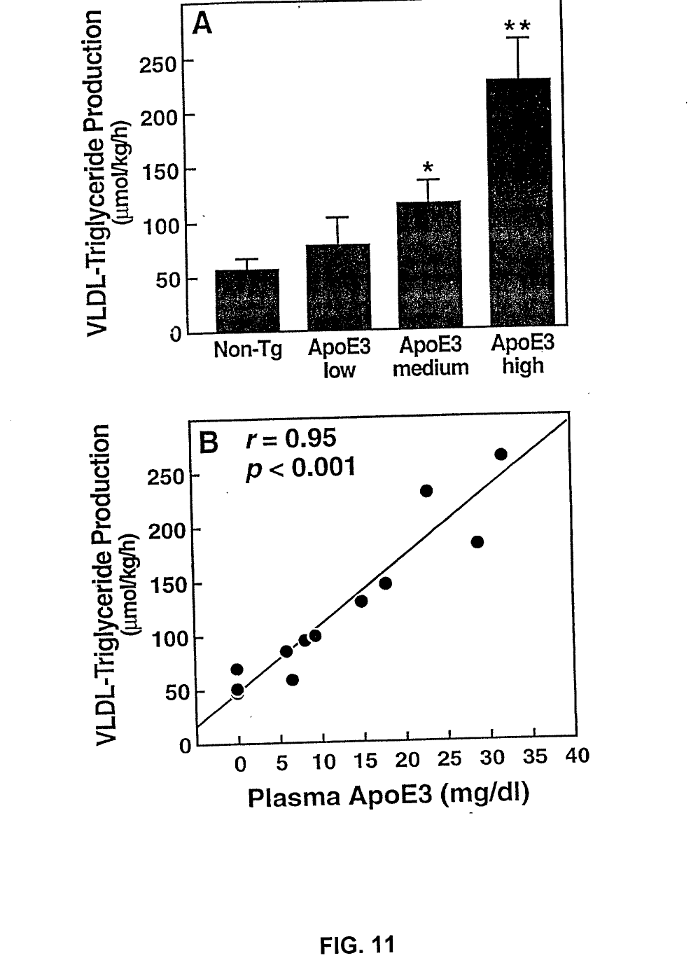

At least three mechanisms could explain the hyperlipidemia associated with apoE3 overexpression: increased VLDL production, impaired VLDL lipolysis, and decreased clearance of apoB-containing lipoproteins. Previously, we demonstrated in transgenic mice that overexpression of apoE3 stimulates hepatic VLDL triglyceride production. However, approximately two-thirds of the apoB secreted by mouse liver is apoB48, whereas the rabbit liver only secretes the apoB100-containing VLDL, raising the possibility that apoE overexpression has differential effects on apoB48 and apoB100 particles. To ascertain if apoE3-overexpressing rabbits have increased hepatic VLDL triglyceride production, we determined VLDL triglyceride production rates in apoE3 transgenic rabbits in which Triton WR1339 was administered intravenously to inhibit lipolysis. The hepatic VLDL triglyceride production rate increased 2- and 4-fold in the medium and high expressers, respectively, but only slightly in the low expressers (FIG. 11A). The VLDL triglyceride production rate correlated positively with plasma apoE3 levels (FIG. 11B). These results suggest that the apoE expression level is an important determinant of VLDL triglyceride production in rabbits.

Next, we determined whether the apoE overexpression-induced changes in triglyceride levels correlated with plasma apoB levels. Apolipoproteins in the VLDL and LDL fractions from nontransgenic and transgenic rabbits were separated by polyacrylamide-SDS gradient gel electrophoresis (VLDL and LDL fractions from the same volume of plasma (200 and 100 μL, respectively) from a single male rabbit from each group were separated on a 3-20% polyacrylamide-SDS gradient gel and stained with Coomassie blue). Densitometric quantitation showed 8- and 25-fold increases in VLDL apoB100 in the medium and high expressers, respectively. Consistent with the similar LDL cholesterol levels in the medium and high expresser transgenic rabbits (FIG. 10E), the LDL apoB levels were also similar, 21 - and 23-fold higher than in the nontransgenic rabbits, respectively. The proportional increase in VLDL and LDL cholesterol and apoB100, together with our previous observation that the mean particle sizes of VLDL and LDL from nontransgenic rabbits and medium expressers are similar, suggests that apoE overexpression may result in a large increase in the number of apoB-containing lipoprotein particles produced by the liver.

Effects of ApoE3 Overexpression on VLDL and IDL Lipolysis in Vitro.

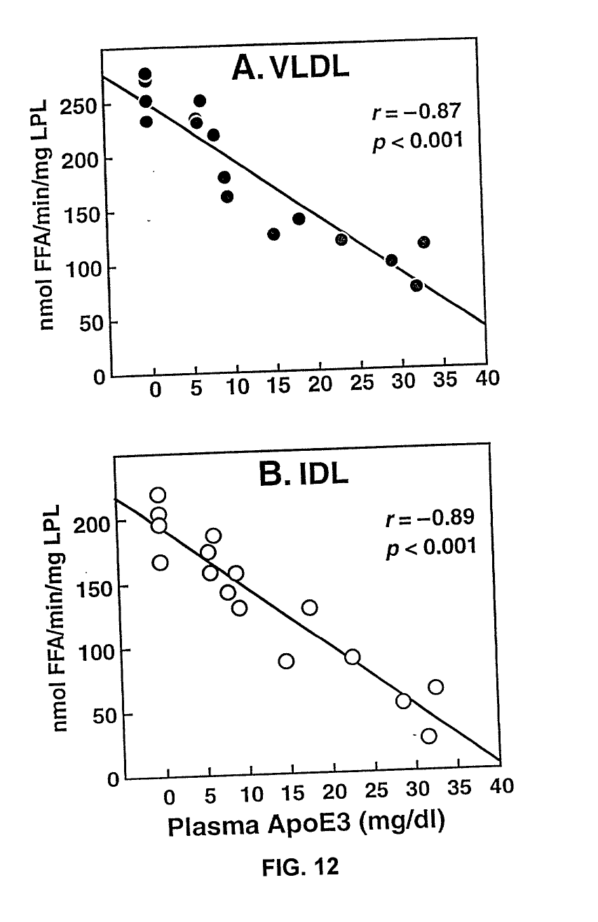

A second mechanism to explain the hyperlipidemia, especially the hypertriglyceridemia associated with apoE3 overexpression, could be an impairment of lipolysis caused by apoE3 accumulation in triglyceride-rich lipoproteins, as previously demonstrated both in vitro and in vivo in apoE3, apoE2, and apoE3-Leiden transgenic mice. Since there was no significant difference in LPL activity of postheparin plasma between nontransgenic and transgenic rabbits, even in the high expressers (data not shown), we assessed the susceptibility of VLDL (d<1.006 g/mL) and IDL (d=1.006-1.02 g/mL) to LPL-mediated lipolysis. The lipolysis of both classes of lipoproteins correlated negatively with plasma apoE3 levels, suggesting a dose-dependent inhibitory effect of apoE3 on LPL-mediated lipolysis (FIG. 12).

Previously, we demonstrated that the impairment of lipolysis caused by apoE accumulation in transgenic mouse VLDL is due mainly to a displacement of apoC-II a well-defined cofactor for LPL activity. To test whether the displacement of apoC-II is also involved in the impairment of lipolysis caused by apoE3 accumulation in transgenic rabbits, we determined the apoC levels in VLDL from nontransgenic and transgenic rabbits by polyacrylamide-SDS gradient gel electrophoresis. Compared with VLDL from controls and apoE3 low expressers, VLDL from high expressers had a much lower content of all of the apoCs (apoC/apoB ratios were 2.76, 1.66, and 0.44 for controls, low expressers, and high expressers, respectively) and a substantially higher content of apoE (apoE/apoB ratios were 0.44, 0.77, and 1.75 for controls, low expressers, and high expressers, respectively). These data indicate that accumulation of apoE3 in transgenic rabbit VLDL either displaces the apoCs from the particles or prevents their association with the particles initially, an effect that may be the primary cause of impaired lipolysis.

Effect of ApoE3 Overexpression on VLDL Clearance in Vivo.

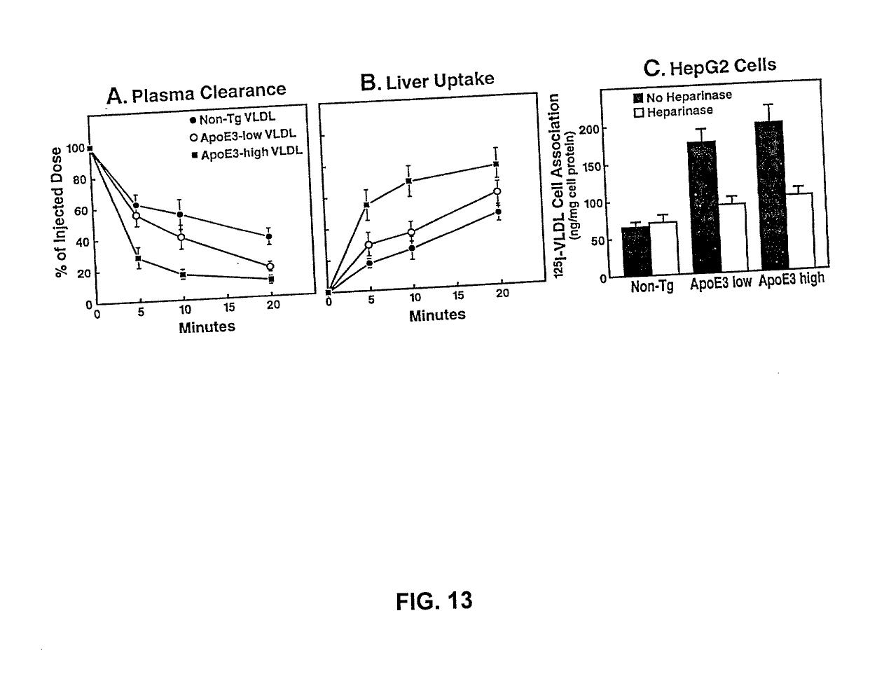

In addition to impaired lipolysis, the accumulation of VLDL in the plasma of apoE3 high expressers raises the possibility that the clearance of VLDL in these transgenic rabbits might be impaired. To address this issue, we determined plasma turnover (FIG. 13A) and liver uptake (FIG. 13B) of ¹²⁵I-labeled control and transgenic rabbit VLDL after intravenous injection into normal mice. The VLDL isolated from apoE3 high expressers was cleared from mouse plasma at a much faster rate than the VLDL from apoE3 low expressers, which were cleared at a faster rate than VLDL from nontransgenics (FIG. 13A). The estimated t_{½} was 4.8, 9.1, and 14 minutes for VLDL from high expressers, low expressers, and nontransgenic rabbits, respectively. The plasma clearance was also reflected in the liver uptake of the labeled lipoproteins: high expresser VLDL>low expresser VLDL>nontransgenic VLDL (FIG. 13B). Consistent with these results, the binding and uptake of ¹²⁵I-VLDL from apoE3 low or high expressers by cultured HepG2 cells was enhanced 2- to 3-fold compared with VLDL from nontransgenics. Moreover, the enhanced cell association of ¹²⁵I-VLDL was nearly abolished by heparinase treatment of the HepG2 cells (FIG. 13C), suggesting the involvement of HSPG in the enhanced clearance of VLDL associated with apoE3 overexpression. These results indicate that overexpression of apoE3 in transgenic rabbits stimulates VLDL clearance, while simultaneously increasing production and inhibiting lipolysis of VLDL.

It is apparent from the above results and discussion that the subject invention provides for improved methods of treating hyperlipidemias, particularly Type IV and Type IIb hyperlipidemias. Also provided by the subject invention are animal models of hyperlipidemia which are useful in the identification of therapeutic agents for hyperlipidemia. As such, the subject invention provides for a significant advance in the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for reducing the plasma level of at least one of VLDL and triglycerides in a host, said method comprising:
administering to said host an effective amount of an agent which at least reduces the amount of plasma active apoE in said host.

2. The method according to claim 1, wherein said agent inhibits apoE.

3. The method according to claim 1, wherein said agent reduces expression of apoE.

4. The method according to claim 1, wherein said apoE is apoE3.

5. A method of treating a host suffering from a disease condition associated with elevated plasma levels of at least one of VLDL and triglycerides, said method comprising:
administering to said host an effective amount of an agent that at least reduces the plasma amount of active apoE in said host.

6. The method according to claim 5, wherein said disease condition is a hyperlipidemia.

7. The method according to claim 6, wherein said hyperlipidemia is Type IV hyperlipidemia.

8. The method according to claim 6, wherein said hyperlipidemia is Type IIb hyperlipidemia.

9. The method according to claim 5, wherein said agent inhibits said apoE.

10. The method according to claim 5, wherein said agent reduces expression of said apoE.

11. The method according to claim 5, wherein said apoE is apoE3.

12. A non-human transgenic animal model of hyperlipidemia, wherein said non-human animal model over-expresses human apo E in a manner sufficient to have a high apoE plasma level, with the proviso that when said non-human transgenic animal model is a lagomorph, said apoE is apoE3.

13. The non-human transgenic animal model according to claim 12, wherein said hyperlipidemia is selected from the group consisting of: (a) hypercholesterolemia; (b) hypertriglyceridemia; and (c) hypertriglyceridemia and hypercholesterolemia.

14. The non-human transgenic animal model according to claim 13, wherein said hyperlipidemia is hypertriglyceridemia.

15. The non-human transgenic animal model according to claim 14, wherein said hyperlipidemia is Type IV hyperlipidemia.

16. The non-human transgenic animal model according to claim 13, wherein said hyperlipidemia is hypertriglyceridemia and hypercholesterolemia.

17. The non-human transgenic animal model according to claim 16, wherein said hyperlipidemia is Type IIb hyperlipidemia.

18. The non-human transgenic animal model according to claim 12, wherein said animal model does not express endogenous apolipoprotein E.

19. The non-human transgenic animal model according to claim 18, wherein said animal is a mouse.

20. A rodent transgenic animal model of hypertriglyceridemia that over-expresses human apolipoprotein E and does not express endogenous apolipoprotein E.

21. The transgenic animal model according to claim 20, wherein said rodent is a mouse.

22. The transgenic animal model according to claim 21, wherein said mouse has plasma human apolipoprotein E levels in excess of about 25 mg/dl.

23. The transgenic animal model according to claim 20, wherein said hypertriglyceridemia is Type IV hyperlipidemia.

24. A lagomorph transgenic animal model of hyperlipidemia that over-expresses human apolipoprotein E3.

25. The transgenic animal model according to claim 24, wherein said animal is a rabbit.

26. The transgenic animal model according to claim 24, wherein said hyperlipidemia is Type IIb hyperlipidemia.

27. The transgenic animal model according to claim 24, wherein said rabbit has plasma human apolipoprotein E3 levels in excess of about 15 mg/dl.

28. A method for screening a compound to determine its effectiveness in treating a disease condition associated with elevated plasma levels of at least one of VLDL and triglycerides, said method comprising:
administering a candidate compound to a non-human animal model according to claim 12; and
determining the effect of said candidate compound on said non-human animal model.

29. The method according to claim 28, wherein said disease condition is hyperlipidemia.

30. The method according to claim 29, wherein said hyperlipidemia is hypertriglyceridemia.

31. The method according to claim 30, wherein said hyperlipidemia is Type IV hyperlipidemia.

32. The method according to claim 29, wherein said hyperlipidemia is hypertriglyceridemia and hypercholesterolemia.

33. The method according to claim 32, wherein said hyperlipidemia is Type IIb hyperlipidemia.

34. A therapeutic compound identified using the screening method of claim 28.

35. A pharmaceutical composition of the therapeutic compound of claim 34.
